(19) United States
(12) Patent Application Publication
(10) Pub. No.: US 2020/0002278 A1
(43) Pub. Date: Jan. 2, 2020

(54) PROCESS FOR PREPARING BRIVARACETAM

(71) Applicant: BEIJING ABLEPHARMTECH CO., LTD., Beijing (CN)

(72) Inventor: Liang MA, Beijing (CN)

(73) Assignee: BEIJING ABLEPHARMTECH CO., LTD., Beijing (CN)

(21) Appl. No.: 16/484,938

(22) PCT Filed: Apr. 23, 2017
(86) PCT No.: PCT/CN2017/081557

§ 371 Date: Aug. 9, 2019

(30) Foreign Application Priority Data
Feb. 24, 2017 (CN) 201710101580.7

(51) Int. Cl. C07D 207/267

(52) U.S. Cl. CPC C07D 207/267

(57) ABSTRACT

The present invention discloses a novel process for preparing Brivaracetam, belonging to the field of chemical synthesis. According to the process, optically pure (R)-4-n-propyl-dihydrofuran-2(3H)-one was used as a starting material, after the steps of ring opening, halogenation, condensation, ring-closing, etc, high-purity Brivaracetam is given. The preparation process has the advantages of easy availability of raw materials, low price, high yield, high optical purity of product, simple reaction conditions and simple operations.

### TECHNICAL FIELD

The present invention belongs to the field of pharmaceutical synthesis, and relates to a novel process for preparing Brivaracetam.

### BACKGROUND ART

Brivaracetam has a structure as shown in formula (I), with chemical name of (2S)-2-((4R)-2-oxo-4-n-propyl-1-pyrrolidinyl) butanamide.

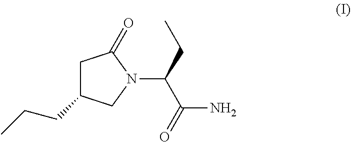

Brivaracetam is a novel high-affinity synaptophysin 2A ligand that inhibits neuronal voltage-dependent sodium channels. It is used for the treatment of partial seizures of refractory epilepsy. In the beginning of 2016, it was approved for marketing in European Union and the United States.

After searching literatures, it is found that there are five synthetic routes of Brivaracetam reported so far.

Benoit M. (JMC 2004, 47, 530-549.) reported a route for preparing Brivaracetam. According to the route, 2(5H)-furanone is used as a starting material, after reaction with n-propylmagnesium bromide, racemic 4-n-propyl-dihydrofuran-2-one is obtained, and then reacted with trimethylsilyl iodide to give open-ring 3-(iodomethyl) hexanoic acid, and after chlorination, 3-(iodomethyl) hexanoyl chloride is obtained, then further reacted with (S)-2-aminobutanamide to give racemic Brivaracetam, after chiral preparation and separation by equipment, finally Brivaracetam is obtained. The specific route is as follows:

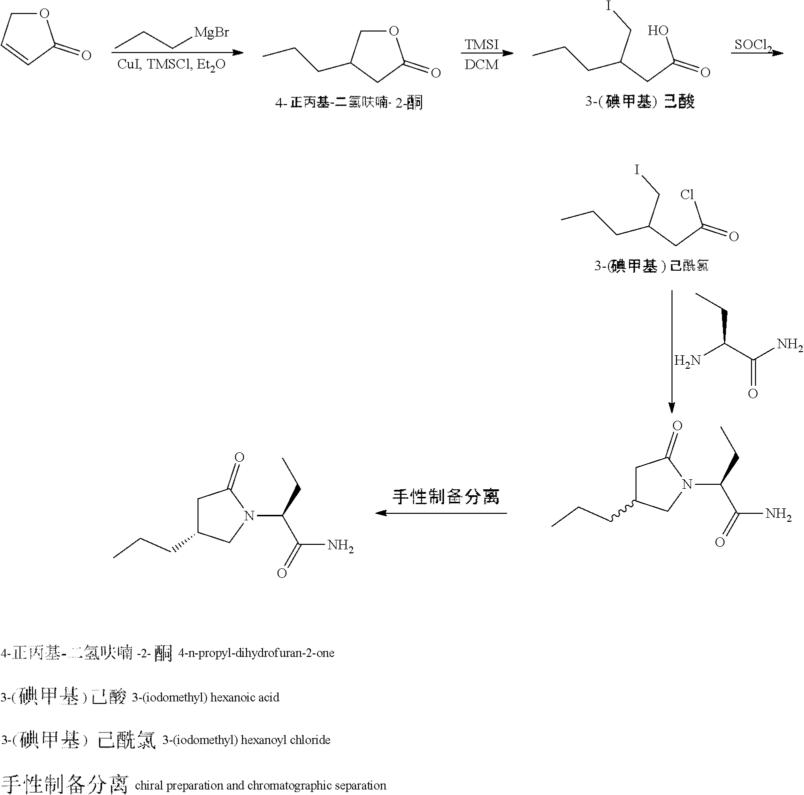

In this route, separation and purification with chiral preparative column is required to get high-purity Brivaracetam, with high production cost and poor industrial feasibility.

The Chinese patent CN101263113B discloses a route for preparing Brivaracetam. According to this route, ethyl 2-hexenoate is used as a staring material, after Michael addition reaction, ethyl 3-nitromethylhexanoate is obtained, after hydrogenation and ring-closing reaction, racemic 4-n-propylpyrrolidone is obtained, then after chiral preparation and chromatographic separation, optically pure (R)-4-n-propylpyrrolidone is given, and then reacted with methyl 2-bromobutyrate to give (2S)-2-(2-oxo-4-n-propyl-1-pyrrolidinyl) methyl butyrate, followed by aminolysis, to give a partially racemized Brivaracetam, and finally a high-purity Brivaracetam is obtained by preparative chromatography. The specific route is as follows:

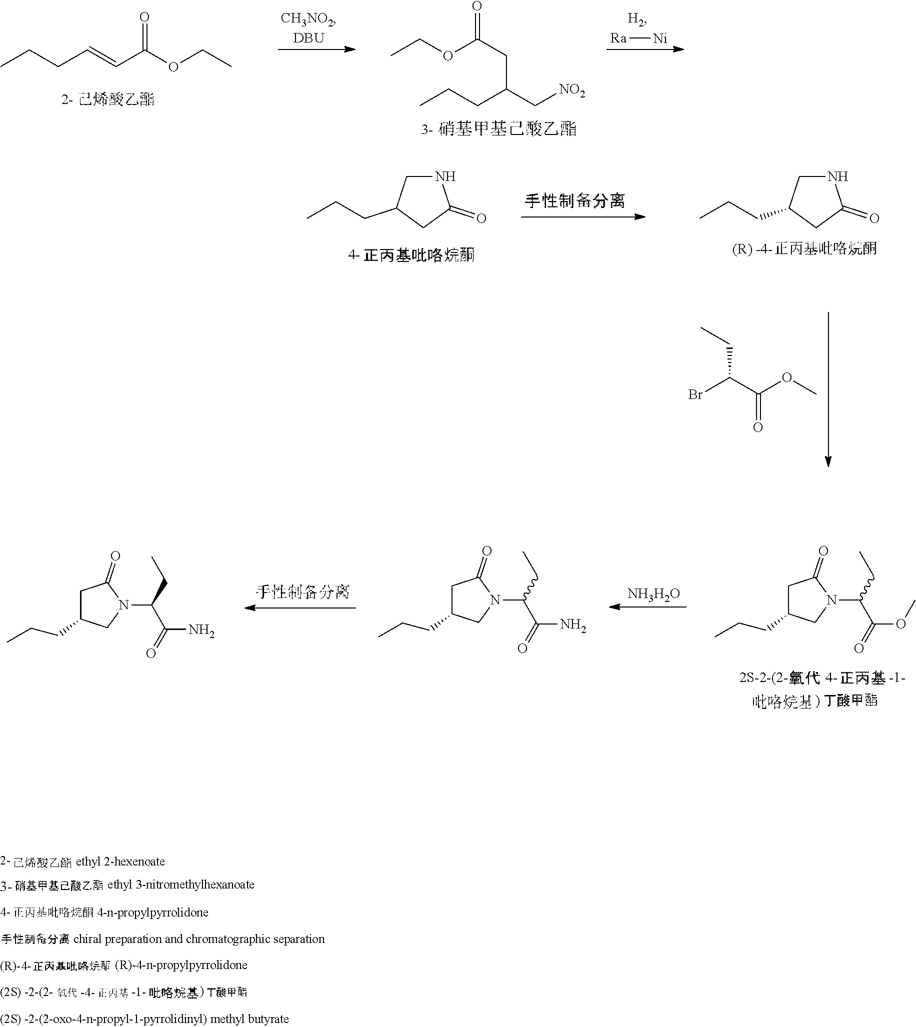

### Chiral Preparation and Chromatographic Separation

In this route, the chiral preparation and chromatographic separation and purification is necessary for the intermediates and final product, with high production cost and poor industrial feasibility.

The patent WO2007065634 discloses a preparation route of Brivaracetam. According to this route, n-pentene is used as a starting material, after asymmetric hydroxylation reaction, (R)-2-hydroxypentanol is obtained, and reacted with sulfoxide chloride to give (4R)-4-propyl-ethylene sulfite, after hydration with ruthenium trichloride and oxidation with sodium periodate, (4R)-4-propyl-ethylene sulfate is given, and then reacted with dimethyl malonate to obtain (S)-6,6-dimethyl-1-propyl-5,7-dioxaspiro[2.5]octane-4,8-dione, followed by reaction with (S)-2-aminobutanamide to give a mixture of a pair of positional isomers, after methylation and decarboxylation, Brivaracetam is obtained. The specific route is as follows:

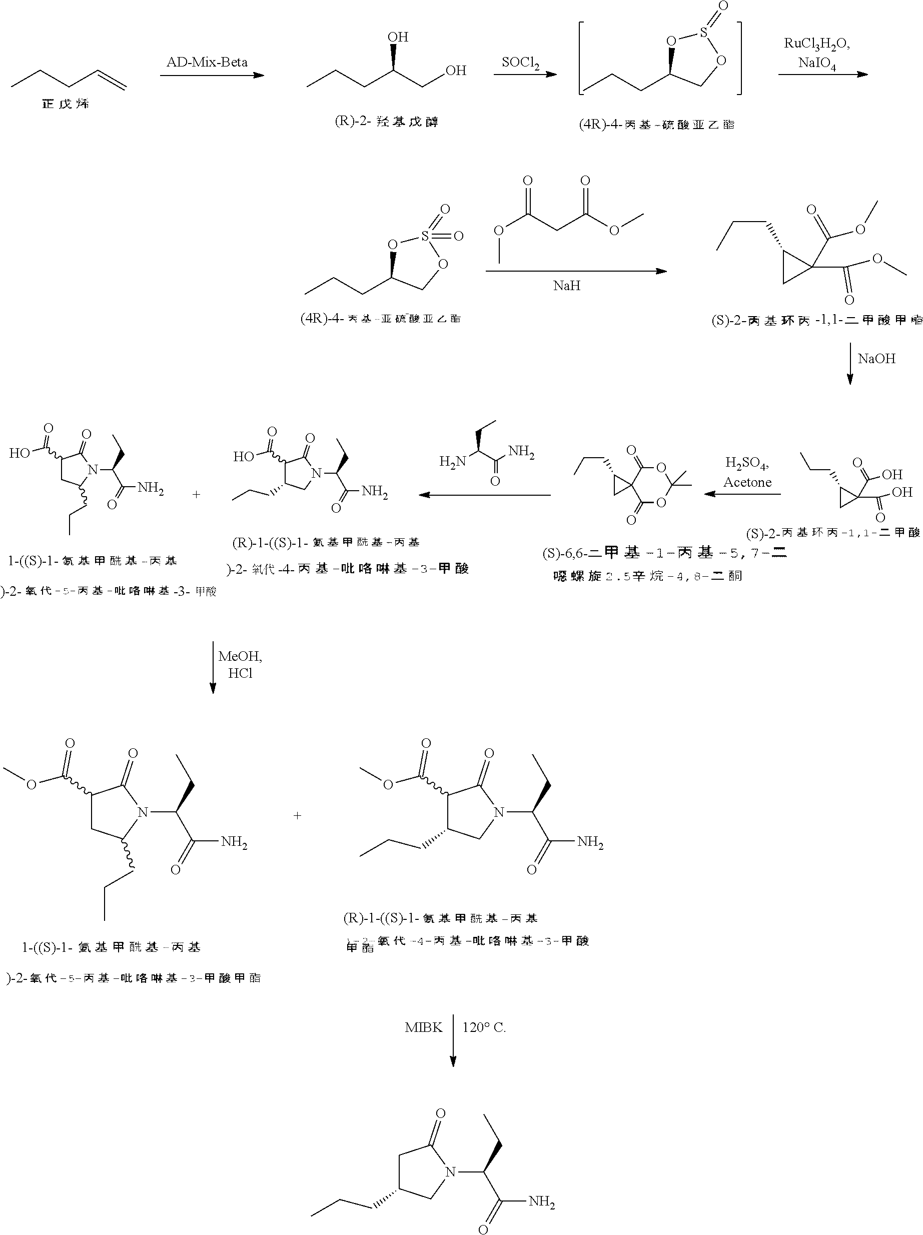

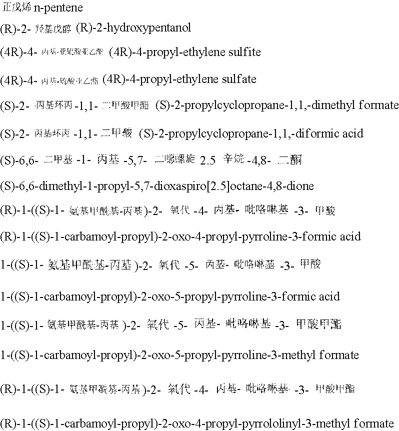

There are two problems for this route. First, the reaction between intermediate (S)-6,6-dimethyl-1-propyl-5,7-dioxaspiro[2.5]octane-4,8-dione and (S)-2-aminobutanamide is not chemoselective, and the product is two positional isomers, which greatly reduces the yield; second, the amide group is present in the Brivaracetam structure, which is easily degraded under high temperature conditions; and in the last step of the route, decarboxylation at a high temperature of 120° C. may produce a large amount of impurities, which brings great difficulty for separation and purification. Therefore, this route is costly and unsuitable for industrial production.

The Chinese patent CN105646319 discloses a preparation route of Brivaracetam. According to this route, diphenyl malonate is used as a starting material, after reacted with (R)-epichlorohydrin, 2-oxo-3-oxabicyclo[3.1.0] hexane-1-phenyl formate is obtained, which is catalyzed by copper iodide and reacted with ethylmagnesium bromide to give 2-oxo-4-propyl-tetrahydrofuran-3-phenyl formate, after decarboxylation at high temperature, (R)-4-propyl-dihydrofuran-2-one is obtained, and after ring-opening with trimethylbromosilane and esterified with methanol, (R) methyl-3-bromomethylhexanoate is obtained, finally a condensation with (S)-2-aminobutanamide is carried out under high temperature to give Brivaracetam. The specific route is as follows:

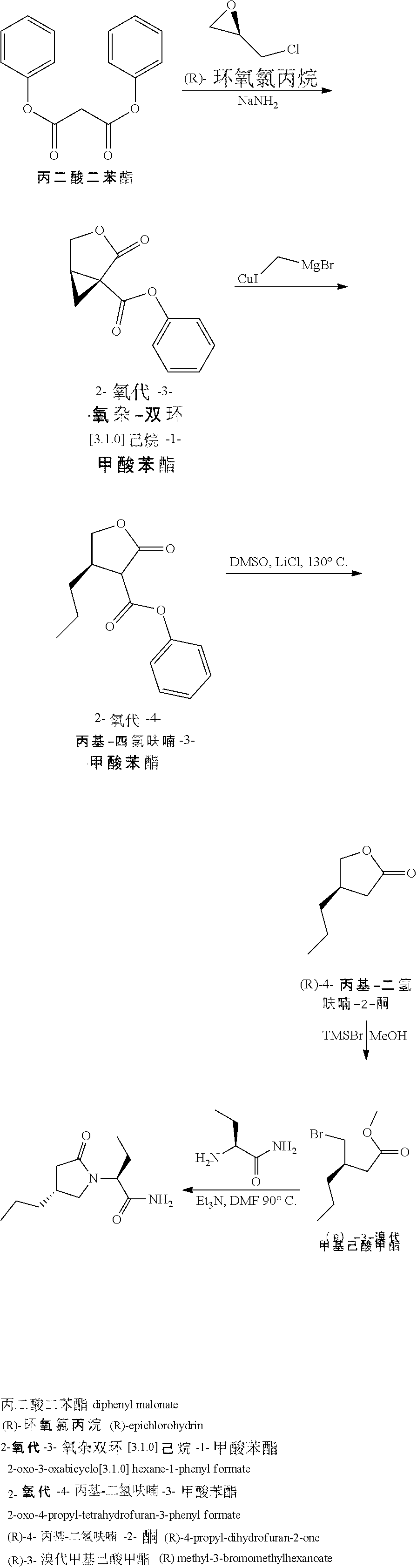

Although chiral separation is not necessary for this route, the last step requires long-term reaction with (S)-2-aminobutanamide under high temperature conditions to give Brivaracetam, which is in conflict with degradation of Brivaracetam under a high temperature and may generate more impurities, bringing great difficulties for purification and separation.

Arnaud Schülé et al (Org. Process Res. Dev. 2016, 20, 1566-1575.) reported a novel route for preparing Brivaracetam. According to this route, racemic 2-propyl-succinic acid 4-tert-butyl ester 1-methyl ester is used as a starting material, after enzymatic resolution, (R)-2-propyl-succinic acid 4-tert-butyl ester 1-methyl ester is given, followed by reduction and ring-closing reaction, (R)-4-propyl-dihydrofuran-2-one is given, then heating and ring-opening in a mixed solution of hydrobromic acid and acetic acid is carried out to give (R)-3-bromomethylhexanoic acid, after ethyl esterification, ethyl (R)-3-bromomethylhexanoate is given, which is finally condensed with (S)-2-aminobutanamide under a high temperature to obtain Brivaracetam. However, enzymatic catalysis is strict and expensive. Further, a solution of hydrogen bromide in acetic acid for preparation of (R)-3-bromomethylhexanoic acid is used under a heating condition of 80° C., and the hydrogen bromide is volatile and highly harmful to equipment and operators. Therefore, this route is costly and not suitable for mass production. The specific route is as follows:

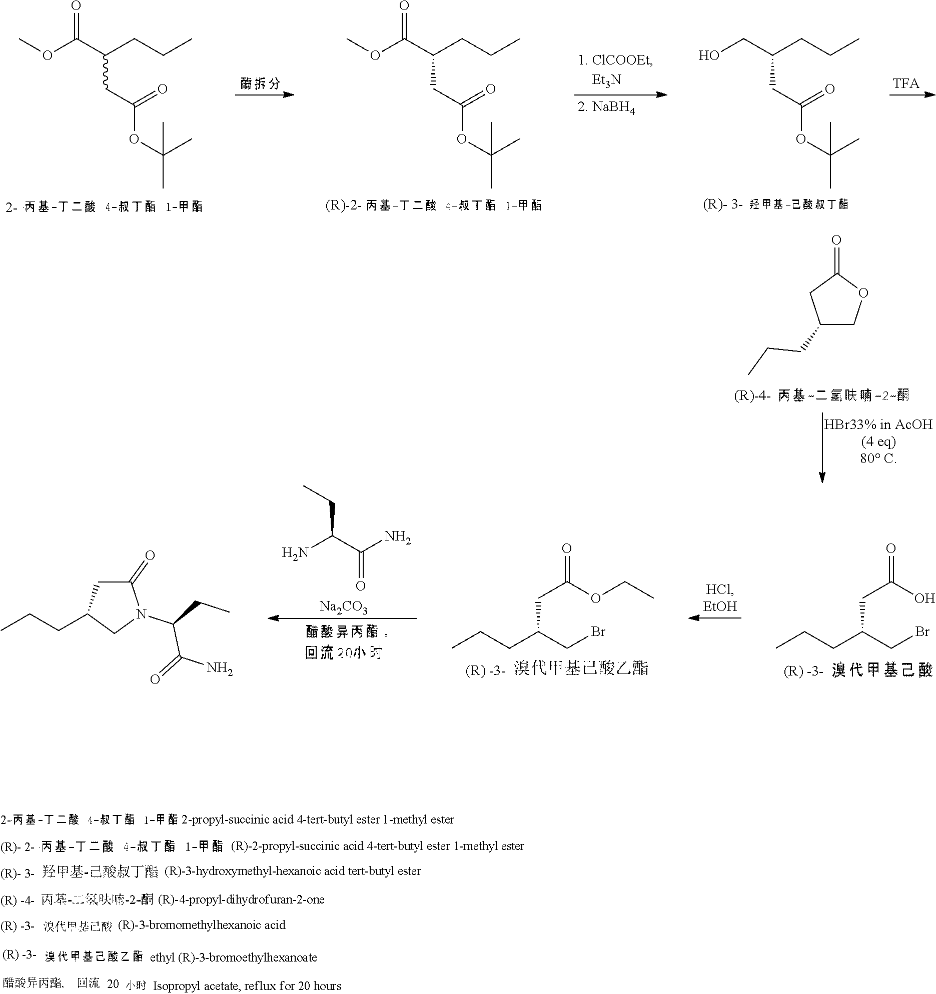

### SUMMARY OF THE INVENTION

In view of the drawbacks of the prior art, it is an object of the present invention to provide a novel process for preparing Brivaracetam. In the present invention, chiral preparation and chromatographic separation steps are not required to directly obtain Brivaracetam with high optical purity, which is more suitable for industrial production. The novel process route has the advantages of easy availableness of starting materials, high reaction yield, simple operation and high chiral purity, thus, it has a broad industrial application prospect.

A novel process for preparing Brivaracetam, comprising the following steps:

1) providing a compound (R)-3-bromomethylhexanoyl halide of formula III,

2) carrying out a condensation reaction of the compound of formula III with (S)-2-aminobutyramide in the presence of an acid-binding agent, to give a compound of formula IV, i.e. (R)-3-bromomethyl-hexanoic acid-[(S)-1-carbamoyl-propyl]-amide,

3) carrying out a substitution reaction of the compound of formula IV in the presence of an alkaline reagent, and a ring-closing reaction, to give the compound of formula I;

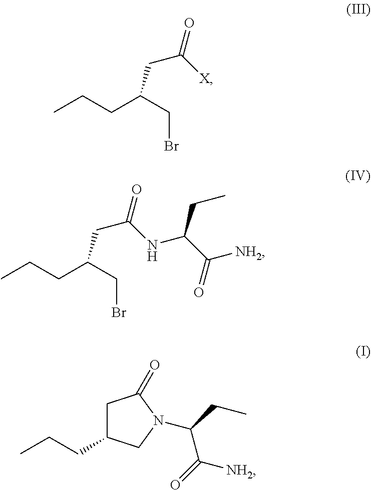

Where, X is selected from chlorine or bromine.

The acid-binding agent is an organic base, and the solvent for the condensation reaction is an aprotic solvent.

Preferably, the acid-binding agent is one or more from triethylamine, pyridine, N,N-diisopropylethylamine, 1,8-diazabicyclo[5.4.0]undec-7-ene (DBU), 1,4-diazabicyclo[2.2.2]octane (DABCO), N,N-dimethylaminopyridine (DMAP), N,N-dimethyl-p-toluidine, and the solvents for the condensation reaction are any one or more from dichloromethane, chloroform, tetrahydrofuran, methyltetrahydrofuran, methyl tert-butyl ether, isopropyl ether, 1,4-dioxane, ethyl acetate, isopropyl acetate, tert-butyl acetate, methyl acetate, ethyl formate.

More preferably, the acid-binding agent is triethylamine or pyridine, and the reaction solvent is tetrahydrofuran.

The molar ratio of the compound of formula III to the acid-binding agent is 1:1-10, the molar ratio of the compound of formula III to (S)-2-aminobutanamide is 1:0.5-5, and the temperature of the condensation reaction is −10 to 50° C.

The molar ratio of the compound of formula III to the acid-binding agent is 1:1-3, the molar ratio of the compound of formula III to (S)-2-aminobutanamide is 1:1.0-2.0, and the temperature of the reaction is −10 to 10° C.

The alkaline reagent is lithium diisopropylamide (LDA), lithium bistrimethylsilylamide (LHDMS), sodium bistrimethylsilylamide (NHDMS), potassium bistrimethylsilylamide (KHDMS), potassium t-butoxide, lithium tert-butoxide, and the solvent for the substitution reaction is an aprotic solvent.

The solvent for the substitution reaction is dichloromethane, chloroform, tetrahydrofuran, methyltetrahydrofuran, methyl tert-butyl ether, isopropyl ether or 1, 4-dioxane.

The alkaline reagent is lithium diisopropylamide (LDA), lithium bistrimethylsilylamide (LHDMS), and the solvent for the substitution reaction is tetrahydrofuran or methyltetrahydrofuran.

The molar ratio of the compound of formula IV to the alkaline reagent is 1:0.9-2.0, and temperature of the substitution reaction is −50-10° C.

The molar ratio of the compound of formula IV to the alkaline reagent is 1:1.0-1.5, and the temperature of the substitution reaction is −30 to −5° C.

The compound of formula III is prepared by the following steps:
(A) the compound of formula V (R)-4-n-propyl-dihydrofuran-2(3H)-one reacts with trimethylbromosilane under the catalysis of anhydrous zinc chloride to give a compound of the formula II, i.e. (R)-3-bromomethyl hexanoic acid,

(B) the compound of formula II reacts with a halogenated agent, to give a compound of formula III;

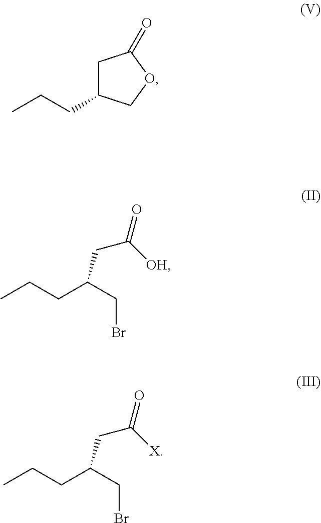

The reaction in the step (A) is carried out in the absence of a solvent or in the presence of an aprotic solvent.

The aprotic solvent is any one or more of dichloromethane, chloroform, toluene, xylene, n-heptane, n-hexane, petroleum ether, cyclohexane, cyclopentane, n-pentane and ethyl acetate.

The aprotic solvent is toluene or n-heptane.

The molar ratio of the compound of the formula V to trimethylbromosilane is 1:1-10, and the molar ratio of the compound of the formula V to anhydrous zinc chloride is 1:0.1-3, the reaction temperature of the step (A) is 20 to 90° C. and the reaction time is 0.5 to 5 hours.

The molar ratio of the compound of the formula V to trimethylbromosilane is 1:2-5, and the molar ratio of the compound of the formula V to anhydrous zinc chloride is 1:0.5-1, the reaction temperature of the step (A) is 60 to 80° C. and the reaction time is 0.5 to 2.0 hours.

The reaction in the step (A) is carried out in the absence of a solvent or in the presence of an aprotic solvent.

The aprotic solvent is any one or more of dichloromethane, chloroform, toluene, xylene, n-heptane, n-hexane, petroleum ether, cyclohexane, cyclopentane, n-pentane, and ethyl acetate, and the halogenated agent is one of thionyl chloride, oxalyl chloride, phosphorus trichloride, phosphorus pentachloride, phosphorus oxychloride, thionyl bromide, oxalyl bromide and phosphorus tribromide.

The aprotic solvent is dichloromethane or toluene, and the halogenated agent is thionyl chloride or oxalyl chloride.

The molar ratio of the compound of formula II to the halogenated agent is 1:1-10, and the reaction temperature of the step (B) is −10 to 50° C.

The molar ratio of the compound of formula II to the halogenated agent is 1:1-4, and the reaction temperature of the step (B) is 0 to 30° C.

In order to achieve the forgoing object, the present invention adopts the following technical solutions:

In the first aspect, the present invention provides a novel process for preparing Brivaracetam, comprising the following steps:

(1) reacting the compound of formula V with trimethylbromosilane under the catalysis of anhydrous zinc chloride to give a compound of formula II, i.e. (R)-3-bromomethylhexanoic acid, with the reaction equation as follows:

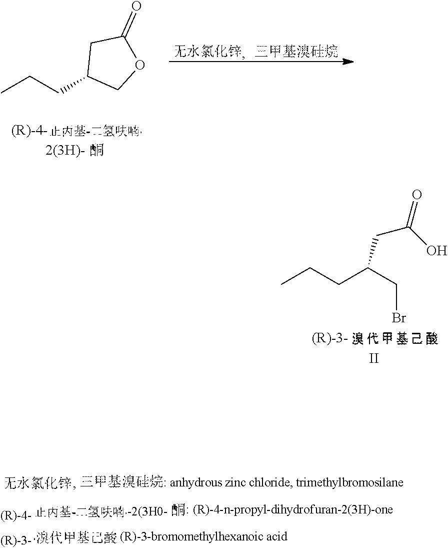

(2) reacting the compound of formula II obtained in the step (1) with a halogenated agent, to give an intermediate of formula III, with the reaction equation as follows:

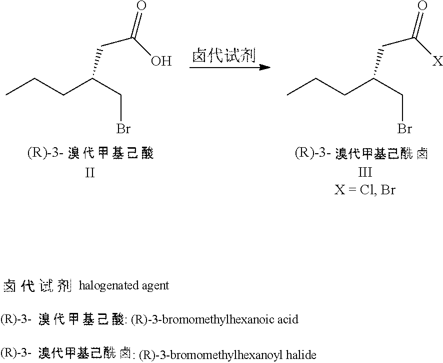

Where, X is selected from chlorine or bromine;

(3) carrying out a condensation reaction of the compound of formula III obtained in the step (2) with (S)-2-aminobutyramide in the presence of an acid-binding agent, to give a compound of formula IV, i.e. (R)-3-bromomethyl-hexanoic acid-[(S)-1-carbamoyl-propyl]-amide, with the reaction equation as follows:

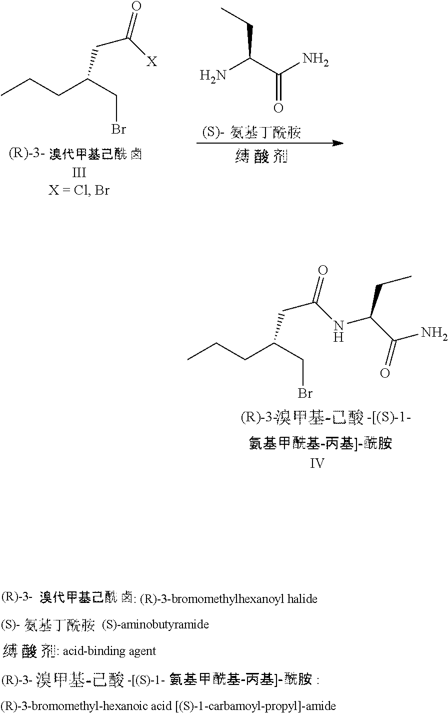

Where, X is selected from chlorine or bromine;

(4) carrying out a substitution reaction of the compound of formula IV obtained in the step (3) in the presence of an alkaline reagent, and a ring-closing reaction, to give the compound of formula I, with the reaction equation as follows:

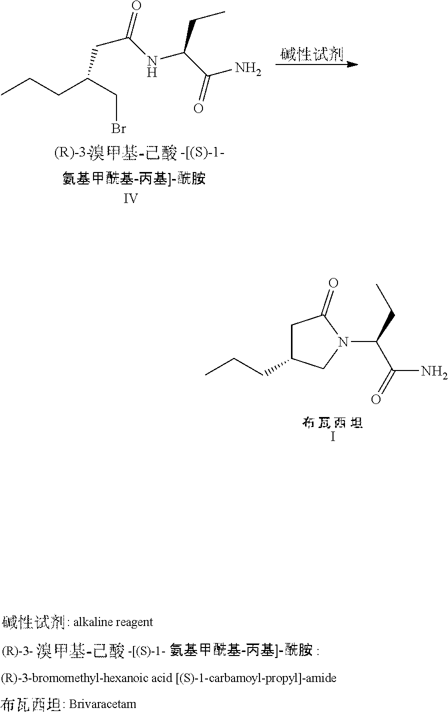

In the present invention, the molar ratio of compound of formula V to trimethylbromosilane in the step (1) is 1:1-10, more preferably from 1:2-5.

Preferably, the molar ratio of compound of formula V to anhydrous zinc chloride is 1:0.1-3, more preferably from 1:0.5-1.

Preferably, the solvent of the reaction in the step (1) is an aprotic solvent, preferably any one or combination of at least two selected from dichloromethane, chloroform, toluene, xylene, n-heptane, n-hexane, petroleum ether, cyclohexane, cyclopentane, n-pentane and ethyl acetate, and more preferably toluene, n-heptane.

Preferably, the reaction of the step (1) can be carried out in the absence of a solvent, that is, trimethylbromosilane acts both as a reactant and as a solvent.

Preferably, the temperature of the reaction in the step (1) is 20-90° C., more preferably 60-80° C.

Preferably, the time of reaction in the step (1) is 0.5 to 5 hours, more preferably 0.5 to 2.0 hours.

In the present invention, the step (1) is carried out by reacting a compound of the formula V with trimethylbromosilane under the catalysis of anhydrous zinc chloride to obtain (R)-3-bromomethylhexanoic acid, with a reaction yield of 80% or more. Studies have shown that the compound of formula V does not substantially react with trimethylbromosilane in the absence of anhydrous zinc chloride. The mechanism shows that the anhydrous zinc chloride, as a Lewis acid, enhances the electropositivity of the carbonyl carbon in the substrate, therefore, it is prone to react when attacked by bromide ion.

In the present invention, the halogenated agent used in the step (2) may be thionyl chloride, oxalyl chloride, phosphorus trichloride, phosphorus pentachloride, phosphorus oxychloride, thionyl bromide, oxalyl bromide, phosphorus tribromide, preferably thionyl chloride or oxalyl chloride.

Preferably, the molar ratio of the compound of formula II to the halogenated agent in the step (2) is 1:1-10, more preferably 1:1-4.

Preferably, the solvent of the reaction in the step (2) is an aprotic solvent, preferably any one or combination of at least two selected from dichloromethane, chloroform, toluene, xylene, n-heptane, n-hexane, petroleum ether, cyclohexane, cyclopentane, n-pentane and ethyl acetate, and more preferably dichloromethane, toluene.

Preferably, the reaction of the step (2) can also be carried out in the absence of a solvent, that is, the halogenated agent acts both as a reactant and as a solvent.

Preferably, the temperature of the reaction in the step (2) is −10 to 50° C., more preferably 0 to 30° C.

In the present invention, the molar ratio of the compound of the formula III to the (S)-2-aminobutanamide in the step (3) is 1:0.5-5, preferably 1:1.0-2.0.

In the present invention, the acid-binding agent used in the step (3) is an organic base, which may be triethylamine, pyridine, N,N-diisopropylethylamine, 1,8-diazabicyclo[5.4.0] undec-7-ene (DBU), 1,4-diazabicyclo[2.2.2]octane (DABCO), N,N-dimethylaminopyridine (DMAP), N,N-dimethyl p-toluidine, more preferably triethylamine or pyridine.

Preferably, the molar ratio of the compound of formula III to the acid-binding agent in the step (3) is 1:1-10, more preferably 1:1-3.

Preferably, the temperature of the reaction in the step (3) is −10-50° C., preferably −10-10° C.

Preferably, the solvent of the reaction in the step (3) is an aprotic solvent, preferably any one or combination of at least two selected from dichloromethane, chloroform, tetrahydrofuran, methyltetrahydrofuran, methyl tert-butyl ether, isopropyl ether, 1,4-dioxane, ethyl acetate, isopropyl acetate, t-butyl acetate, methyl acetate, ethyl formate, and more preferably tetrahydrofuran.

In the present invention, the alkaline reagent in the step (4) is lithium diisopropylamide (LDA), lithium bistrimethylsilylamide (LHDMS), sodium bistrimethylsilylamide (NHDMS), potassium bis-trimethylsilylamino (KHDMS), potassium t-butoxide, lithium t-butoxide, more preferably LHDMS or LDA.

Preferably, the molar ratio of the compound of formula IV to the alkaline agent in the step (4) is 1:0.9-2.0, more preferably 1:1.0-1.5.
Preferably, the reaction solvent in the step (4) is an aprotic solvent, specifically dichloromethane, chloroform, tetrahydrofuran, methyltetrahydrofuran, methyl tert-butyl ether, isopropyl ether, 1,4-dioxane, more preferably tetrahydrofuran or methyltetrahydrofuran.
Preferably, the temperature of the reaction in the step (4) is −50 to 10° C., more preferably −30 to −5° C.

In the present invention, the compound of the formula IV and the base form a nitrogen anion at a low temperature in the step (4), to attack the halogenated alkane, after ring-closing, the compound of the formula I is obtained. The compound of formula I contains two chiral centers, and the configuration of the 2-position carbon is prone to racemization under alkaline conditions, and the temperature has a significant influence on the racemization. Studies have shown that, when the reaction temperature is controlled below 0° C., the amount of 2-position racemized impurities can be well controlled, to get the product with high chiral purity. The product purity can reach 99% or more, and the impurities with 2-position racemization can be controlled within 0.15%.

### DETAILED DESCRIPTION OF THE PREFERRED EMBODIMENTS

The present invention is further described in detail below with reference to the embodiments.

The starting reagents used below are either commercially available or prepared by the methods in the literatures.

The general reaction is as follows:

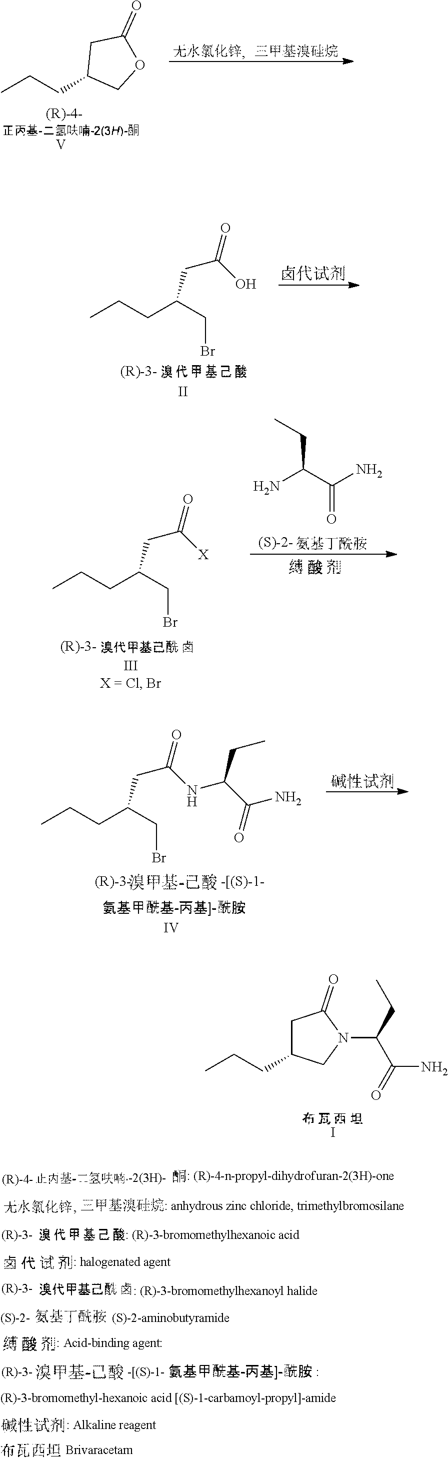

### Embodiment 1: Synthesis of (R)-3-bromomethylhexanoic Acid (Toluene as a Solvent)

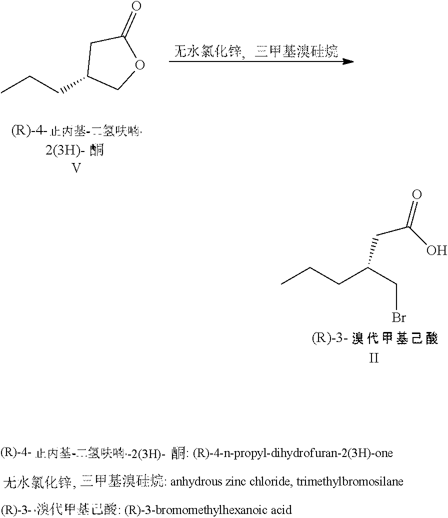

Operation Procedure:

In a 250 mL three-necked flask, dry toluene (60 mL) was added, and (R)-4-n-propyl-dihydrofuran-2(3H)-one (12.8 g, 0.1 mol, 1 eq) and anhydrous zinc chloride (6.8 g, 0.05 mol, 0.5 eq) were added sequentially, then trimethylbromosilane (61.2 g, 0.4 mol, 4 eq) was added dropwise while stirring, after the addition, the mixture was heated to 70˜80° C. and the reaction was conducted for 1 hour. TLC was used to detect the disappearance of (R)-4-n-propyl-dihydrofuran-2(3H)-one, then heating was stopped and cooling was started. Water (100 mL) was added dropwise at the temperature of 0˜10° C. to quench the reaction, then the liquid was separated, the organic phase was washed with water (100 mL×2) and then washed with saturated sodium chloride solution (100 mL). The organic phase was collected and dried over anhydrous sodium sulfate (10 g) for 2 hours, filtered, and concentrated until dryness to give the target compound as a light yellow oily substance (18.1 g, yield 86.2%), and then used in the subsequent step directly.

¹H NMR (400 MHz, Chloroform-d) δ 10.94 (s, 1H), 3.58 (dd, J=10.3, 4.1 Hz, 1H), 3.50 (dd, J=10.3, 5.2 Hz, 1H), 2.57 (dd, J=16.4, 7.3 Hz, 1H), 2.41 (dd, J=16.5, 6.0 Hz, 1H), 2.26-2.13 (m, 1H), 1.54-1.27 (m, 4H), 0.93 (t, J=6.8 Hz, 3H). [α]_{D}¹⁷+4.4° (c=0.9 g/100 mL, CHCl₃).

### Embodiment 2: Synthesis of (R)-3-bromomethylhexanoic Acid (n-Heptane as a Solvent)

Operation Procedure:

In a 250 mL three-necked flask, dry n-heptane (60 mL) was added at room temperature, and (R)-4-n-propyl-dihydrofuran-2(3H)-one (12.8 g, 0.1 mol, 1 eq) and anhydrous zinc chloride (6.8 g, 0.05 mol, 0.5 eq) were added sequentially, and then trimethylbromosilane (61.2 g, 0.4 mol, 4 eq) was added dropwise while stirring, after the addition, the mixture was heated to 70˜80° C. and the reaction was conducted for 1 hour. TLC was used to detect the disappearance of (R)-4-n-propyl-dihydrofuran-2(3H)-one, then heating was stopped and cooling was started. Water (100 mL) was added dropwise at the temperature of 0˜10° C. to quench the reaction, then the liquid was separated, the organic phase was washed with water (100 mL×2) and then washed with saturated sodium chloride solution (100 mL). The organic phase was collected and dried over anhydrous sodium sulfate (10 g) for 2 hours, filtered, and concentrated until dryness to give the target compound as a light yellow oily substance (19.4 g, yield 92.4%), and then used in the subsequent step directly.

### Embodiment 3: Synthesis of (R)-3-bromomethylhexanoyl chloride

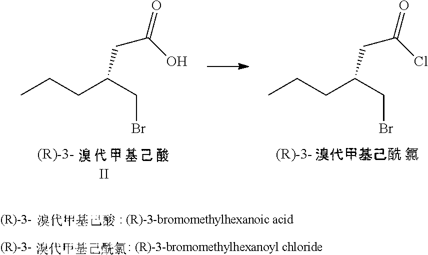

### Operation Procedure:

In a 250 mL three-necked flask, dichloromethane (100 mL) was added, and (R)-3-bromomethylhexanoic acid (19.0 g, 0.09 mol, 1 eq) was added, then thionyl chloride (32.1 g, 0.27 mol, 3eq) was added dropwise while stirring. After the addition, the reaction was conducted at room temperature while stirring. TLC was used to detect the disappearance of the starting materials, and then the reaction was stopped, and the mixture was concentrated to dryness, to give the target compound as yellow oily substance (21.4 g, yield 104.5%), and then used in the subsequent step directly.

¹H NMR (400 MHz, Chloroform-d) δ 3.58 (ddd, J=18.6, 10.5, 3.9 Hz, 1H), 3.52-3.42 (m, 1H), 3.20-2.87 (m, 1H), 2.73-2.36 (m, 1H), 2.33-2.14 (m, 1H), 1.53-1.26 (m, 4H), 0.98-0.89 (m, 3H).

### Embodiment 4: Synthesis of (R)-3-bromomethyl-hexanoic acid-[(S)-1-carbamoyl-propyl]-amide

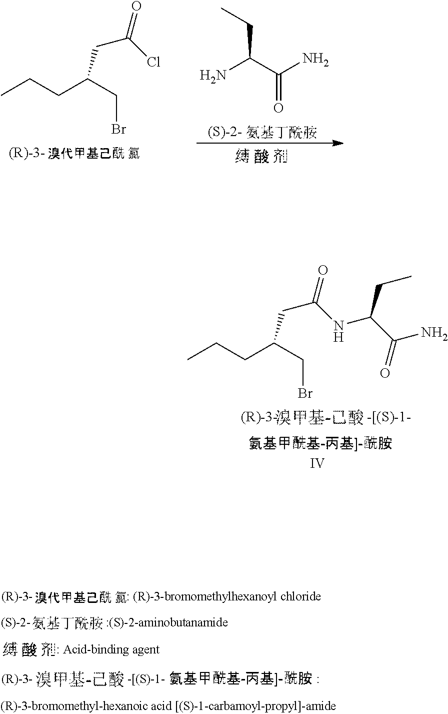

Operation Procedure:

In a 500 mL three-necked flask, tetrahydrofuran (100 mL), triethylamine (18.2, 0.18 mol, 2 eq) and (S)-2-aminobutanamide (11.2 g, 0.11 mol, 1.2 eq) were added at room temperature. After the dissolution, the temperature was lowered to 0 to 10° C., and (R)-3-bromomethylhexanoyl chloride (content: 95%, 21.4 g, 0.09 mol, 1 eq) was added dropwise, and after the addition, the reaction was conducted for 1-2 hours at a constant temperature. After completion of the reaction, water (300 mL) was added to the reaction system, and stirred to separate out the solid, filtered, and the filter cake was rinsed with water. The filter cake was collected and dried at 45° C. by forced air for 5 hours. The dried solid was collected to give the target compound as a white solid (19.6 g, yield 74.3%).

¹H NMR (400 MHz, Chloroform-d) δ 6.57-6.25 (m, 2H), 5.73 (s, 1H), 4.46 (td, J=7.5, 6.1 Hz, 1H), 3.53 (d, J=3.9 Hz, 2H), 2.40-2.24 (m, 2H), 2.24-2.17 (m, 1H), 1.91 (ddd, J=13.7, 7.6, 6.2 Hz, 1H), 1.69 (dt, J=14.2, 7.2 Hz, 1H), 1.35 (dtdd, J=22.2, 11.9, 8.5, 5.8 Hz, 4H), 0.98 (t, J=7.4 Hz, 3H), 0.92 (t, J=6.7 Hz, 3H). MS(ESI): m/z 293.0[M+H]; [α]_{D}¹⁷ −57.5° (c=1.0 g/100 mL, CHCl₃).

### Embodiment 5: Synthesis of Brivaracetam

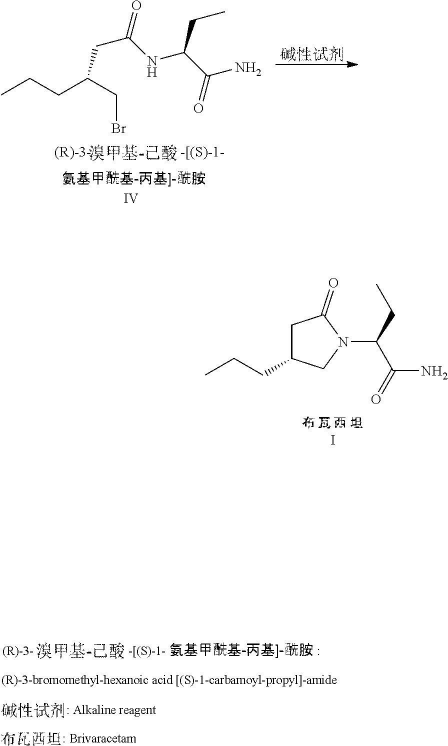

### Operation Procedure:

In a three-necked flask, tetrahydrofuran (130 mL) was added, and (R)-3-bromomethyl-hexanoic acid-[(S)-1-carbamoyl-propyl]-amide (13.0 g, 44.3 mmol, 1 eq) was added, cooled to −30˜−20° C., and then 1.0 M LHMDS (53.2 mL, 53.2 mmol, 1.2 eq) was added dropwise. After the addition, the temperature was raised to −10˜−5° C. for 1 hour. TLC was used to detect the disappearance of reaction substances. The reaction was quenched by adding saturated ammonium chloride solution (100 mL). The liquid was separated, and the organic phase was washed with water (30 mL) and washed with saturated sodium chloride solution (30 mL), and dried over anhydrous sodium sulfate (10 g) for 2 hours, filtered. The filtrate was concentrated to dryness under a reduced pressure at 40° C., then isopropyl ether (30 mL) was added to the concentrate and stirred for 2 hours to separate out a solid, suction filtered and the filter cake was rinsed with isopropyl ether. The solid was collected and dried by forced air at 45° C. for 4 hours, to give Brivaracetam as a white solid (6.6 g, yield 70.2%).

¹H NMR (400 MHz, DMSO-d₆) δ 7.33 (s, 1H), 6.99 (s, 1H), 4.30 (dd, J=10.3, 5.4 Hz, 1H), 3.37 (t, J=8.7 Hz, 1H), 3.11 (dd, J=9.5, 7.0 Hz, 1H), 2.38 (dd, J=16.1, 8.5 Hz, 1H), 2.23 (p, J=7.6 Hz, 1H), 1.98 (dd, J=16.1, 8.0 Hz, 1H), 1.78 (dp, J=13.9, 7.2 Hz, 1H), 1.56 (ddt, J=17.5, 14.3, 7.4 Hz, 1H), 1.45-1.21 (m, 4H), 0.88 (t, J=7.1 Hz, 3H), 0.77 (t, J=7.3 Hz, 3H). MS(ESI): m/z 213.2 [M+H]⁺; [α]_{D}²⁰ −62.0° (c=1.0 g/100 mL, MeOH).

1. A novel process for preparing Brivaracetam, comprising the following steps:
1) providing a compound (R)-3-bromomethylhexanoyl halide of formula III,
2) carrying out a condensation reaction of the compound of formula III with (S)-2-aminobutyramide in the presence of an acid-binding agent, to give a compound of formula IV, i.e. (R)-3-bromomethyl-hexanoic acid-[(S)-1-carbamoyl-propyl]-amide,
3) carrying out a substitution reaction of the compound of formula IV in the presence of an alkaline reagent, and a ring-closing reaction, to give the compound of formula I; 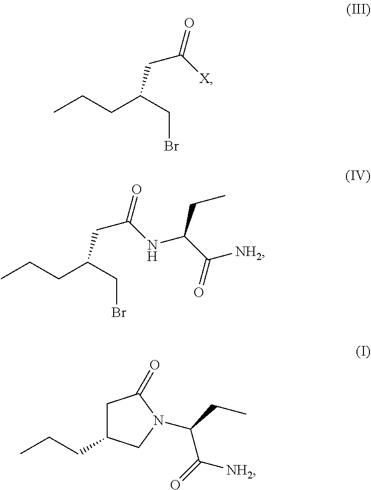
where, X is selected from chlorine or bromine.

2. The preparation process according to claim 1, wherein the acid-binding agent is an organic base, and the solvent for the condensation reaction is an aprotic solvent.

3. The preparation process according to claim 2, wherein the acid-binding agent is one or more from triethylamine, pyridine, N,N-diisopropylethylamine, 1,8-diazabicyclo[5.4.0]undec-7-ene, 1,4-diazabicyclo [2.2.2]octane, N,N-dimethylaminopyridine, N,N-dimethyl-p-toluidine, and the solvents for the condensation reaction are any one or more from dichloromethane, chloroform, tetrahydrofuran, methyltetrahydrofuran, methyl tert-butyl ether, isopropyl ether, 1,4-dioxane, ethyl acetate, isopropyl acetate, tert-butyl acetate, methyl acetate, ethyl formate.

4. The preparation process according to claim 3, wherein the acid-binding agent is triethylamine or pyridine, and the solvent for condensation reaction is tetrahydrofuran.

5. The preparation process according to claim 1, wherein the molar ratio of the compound of formula III to the acid-binding agent is 1:1-10, the molar ratio of the compound of formula III to (S)-2-aminobutanamide is 1:0.5-5, and the temperature of the condensation reaction is −10 to 50° C.

6. The preparation process according to claim 5, wherein the molar ratio of the compound of formula III to the acid-binding agent is 1:1-3, the molar ratio of the compound of formula III to (S)-2-aminobutanamide is 1:1.0-2.0, and the temperature of the reaction is −10 to 10° C.

7. The preparation process according to claim 1, wherein the alkaline reagent is lithium diisopropylamide, lithium bistrimethylsilylamide, sodium bistrimethylsilylamide, potassium bistrimethylsilylamide, potassium t-butoxide, lithium tert-butoxide, and the solvent for the substitution reaction is an aprotic solvent.

8. The preparation process according to claim 7, wherein the solvent for the substitution reaction is dichloromethane, chloroform, tetrahydrofuran, methyltetrahydrofuran, methyl tert-butyl ether, isopropyl ether or 1, 4-dioxane.

9. The preparation process according to claim 8, wherein alkaline reagent is lithium diisopropylamide, lithium bistrimethylsilylamide and the solvent for the substitution reaction is tetrahydrofuran or methyltetrahydrofuran.

10. The preparation process according to claim 7, the molar ratio of the compound of formula IV to the alkaline reagent is 1:0.9-2.0, and the temperature of the substitution reaction is −50 to 10° C.

11. The preparation process according to claim 10, wherein the molar ratio of the compound of formula IV to the alkaline reagent is 1:1.0-1.5, and the temperature of the substitution reaction is −30 to −5° C.

12. The preparation process according to claim 1, wherein the compound of formula III is prepared by the following steps:
(A) the compound of formula V reacts with trimethylbromosilane under the catalysis of anhydrous zinc chloride to give a compound of the formula II, i.e. (R)-3-bromomethylhexanoic acid,
(B) the compound of formula II reacts with a halogenated agent, to give a compound of formula III;
(V), (II), (III).

13. The preparation process according to claim 12, wherein the reaction in the step (A) is carried out in the absence of a solvent or in the presence of an aprotic solvent.

14. The preparation process according to claim 13, wherein the aprotic solvent is any one or more of dichloromethane, chloroform, toluene, xylene, n-heptane, n-hexane, petroleum ether, cyclohexane, cyclopentane, n-pentane and ethyl acetate.

15. The preparation process according to claim 14, wherein the aprotic solvent is toluene or n-heptane.

16. The preparation process according to claim 12, wherein the molar ratio of the compound of the formula V to trimethylbromosilane is 1:1-10, and the molar ratio of the compound of the formula V to anhydrous zinc chloride is 1:0.1-3, the reaction temperature of the step (A) is 20 to 90° C. and the reaction time is 0.5 to 5 hours.

17. The preparation process according to claim 16, wherein molar ratio of the compound of the formula V to trimethylbromosilane is 1:2-5, and the molar ratio of the compound of the formula V to anhydrous zinc chloride is 1:0.5-1, the reaction temperature of the step (A) is 60 to 80° C. and the reaction time is 0.5 to 2.0 hours.

18. The preparation process according to claim 12, wherein the reaction in the step (B) is carried out in the absence of a solvent or in the presence of an aprotic solvent.

19. The preparation process according to claim 18, wherein the aprotic solvent is any one or more of dichloromethane, chloroform, toluene, xylene, n-heptane, n-hexane, petroleum ether, cyclohexane, cyclopentane, n-pentane, and ethyl acetate, and the halogenated agent is one of thionyl chloride, oxalyl chloride, phosphorus trichloride, phosphorus pentachloride, phosphorus oxychloride, thionyl bromide, oxalyl bromide and phosphorus tribromide.

20. The preparation process according to claim 19, wherein the aprotic solvent is dichloromethane or toluene, and the halogenated agent is thionyl chloride or oxalyl chloride.

21. The preparation process according to claim 18, wherein the molar ratio of the compound of formula II to the halogenated agent is 1:1-10, and the reaction temperature of the step (B) is −10 to 50° C.

22. The preparation process according to claim 21, wherein the molar ratio of the compound of formula II to the halogenated agent is 1:1-4, and the reaction temperature of the step (B) is 0 to 30° C.
